(19) United States
(12) Patent Application Publication
(10) Pub. No.: US 2021/0069525 A1
(43) Pub. Date: Mar. 11, 2021

(54) PHOTOBIOMODULATION WEARABLE FOR PERFORMANCE ENHANCEMENT

(71) Applicant: DMC Limited L.C., Sheridan, WY (US)

(72) Inventors: Daniel Gerard Schupp, Detroit Lakes, MN (US); Charles Joseph Schupp, Minnetrista, MN (US); Max David Disse, Hummelstown, PA (US); John Mario Sartori, Minnetrista, MN (US); Andrew Schupp, Minneapolis, MN (US)

(21) Appl. No.: 17/053,675

(22) PCT Filed: May 7, 2019
(86) PCT No.: PCT/US2019/031169

§ 371 Date: Nov. 6, 2020

Related U.S. Application Data
(60) Provisional application No. 62/668,062, filed on May 7, 2018.

(51) Int. Cl. A61N 5/06; A41D 1/00

(52) U.S. Cl. CPC A61N 5/0613; A41D 1/002; A61N 2005/0645; A61N 2005/066; A61N 2005/0628; A61N 2005/0663

(57) ABSTRACT

A method includes applying a substrate containing light emitting devices to an individuals body part and controlling the light emitting devices to deliver athletic performance enhancing light to the body part, wherein the light has a wavelength in the range of 400 nm to 1300 nm and an irradiance of from 2 mW-600 mW/cm2.

### RELATED APPLICATION

This application claims priority to U.S. Provisional Application Ser. No. 62/668,062 (entitled Wearable Photobiomodulation Technology for Performance Enhancement, filed May 7, 2018) which is incorporated herein by reference.

### BACKGROUND

Light has been used in various methods of medical treatment, such as to enhance healing of burns and other medical conditions. Light therapy has included exposure to daylight or to specific wavelengths of light using polychromatic polarized light, lasers, light-emitting diodes, fluorescent lamps, dichroic lamps or very bright, full-spectrum light. The light is administered for a prescribed amount of time and, in some cases, at a specific time of day.

One common use of the term is associated with the treatment of skin disorders, chiefly psoriasis, acne vulgaris, eczema and neonatal jaundice.

Light therapy which strikes the retina of the eyes is used to treat diabetic retinopathy and also circadian rhythm disorders such as delayed sleep phase disorder and can also be used to treat seasonal affective disorder, with some support for its use also with non-seasonal psychiatric disorders.

Photobiomodulation (PBM) has been proposed, researched and utilized for treatment of various disease states with proven benefits enhancing healing in a variety of organ systems including hair regrowth, myriad of skin conditions, wound healing and improved perfusion in experimental models of ischemia.

More recently, enhancement of normal tissue bioenergetic and cellular function has been proposed and researched with proven benefits for both performance and recovery utilized before and/or after in a variety of laboratory exercise tests.

There are several devices on the market utilizing both LED and low-level laser (LLL) therapy as either a hand held or total body enclosure treatment system, and/or for healing/treatment of diseases or disease states as well as athletic performance.

### SUMMARY

Light producing mechanisms are embedded in, or associated with, sportswear and clothing material and directed at specific muscle groups/skin sites to stimulate on a cellular, subcellular and systemic level the enhancement of muscular and exercise/activity performance.

### BRIEF DESCRIPTION OF THE DRAWINGS

FIG. 1 is a block diagram of a system using photobiomodulation for performance enhancement according to an example embodiment.

FIG. 2 is a block diagram illustrating a wearable item having electronics and light actuators according to an example embodiment.

FIG. 3 is a side, front, and back inside-out view of wearable shorts incorporating a system for performance enhancement according to an example embodiment.

FIG. 4 illustrates a wearable sleeve being worn and incorporating a system for performance enhancement according to an example embodiment.

FIG. 5 illustrates an inside-out view of a wearable shirt incorporating a system for performance enhancement according to an example embodiment.

FIG. 6 illustrates a wearable sock being worn and incorporating a system for performance enhancement according to an example embodiment.

FIG. 7 illustrates a wearable pair of pants/tights being worn and incorporating a system for performance enhancement according to an example embodiment.

FIG. 8 is block cross sectional representation of multiple different wearable arrangements of light sources according to an example embodiment.

FIGS. 9A and 9B are inside-out front and back schematic line diagrams of a prototype pair of shorts incorporating a system for performance enhancement according to an example embodiment.

FIGS. 10A, 10B, 10C, 10D, 10E, 10F, and 10G are a flowchart illustrating a computer implemented method 1000 use and operation of a wearable, such as a garment for enhancing performance according to an example embodiment.

FIG. 10H is a block diagram showing the arrangement of FIGS. 10A, 10B, 10C, 10D, 10E, 10F, and 10G with respect to each other.

FIG. 11 is a block diagram illustrating circuitry for controlling lights to enhance performance of selected muscle groups and performing methods according to an example embodiment.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

Various devices and method are described herein that utilize photobiomodulation (PBM) in performance enhancing sportswear and external devices utilizing arrays of light emitting diodes (LEDs), low level laser light (LLL), or multitudes of both functioning at specific bandwidths with pulsed or continuous electromagnetic energy in the visible and near infrared portion of the light spectrum for all types of physical endeavors. Light can act on different mechanisms within cellular tissue to stimulate or suppress biological activity in a process commonly referred to as photobiomodulation (PBM). PBM involves the use of visible to near infrared light (NIR) (400-1300 nm) produced by a laser or a non-coherent light source applied to the surface of the body and has been shown to produce beneficial effects, most recently in activity and sports athletic performance.

PBM utilizes light with a suitable intensity, energy, and wavelengths, without significantly causing damage to the cells. Some photo-acceptors, such as water or hemoglobin, are ubiquitous and absorb light to such a degree that little or no penetration of light energy into a tissue occurs. For example, water absorbs light most willingly above approximately 1300 nanometers. Thus, energy in this range has little ability to penetrate tissue due to the water content. However, water is more transparent in wavelengths between 400 and 1300 nanometers. Another example is hemoglobin, which absorbs heavily in the region between 300 and 670 nanometers but is reasonably transparent above approximately 670 nanometers.

Based on these broad assumptions, one can define a “spectral window” into the body. Within the window, there are certain wavelengths that are more or less likely to penetrate and target a specific chromophore and resultant target effect. The mechanism of PBM at the cellular level has been ascribed to the activation of mitochondrial respiratory chain components resulting in stabilization of metabolic function. A growing body of evidence suggests that the mitochondrial enzyme cytochrome C oxidase (CCO) is a key photo-acceptor of light in the far red to near infrared spectral range. These specific chromophores or photo-acceptors in the target tissue, most notably CCO, and nitric oxide (NO) may have unique interdependent roles in controlling cellular energy production and maintenance of cellular redox status.

Under certain conditions such as hypoxia or cellular fatigue, NO is produced, which has an inhibitory effect on the electron transport chain (ETC) by binding to and deactivating CCO which is the final electron acceptor of the ETC. Light therapy has been shown to dissociate bound NO from CCO and other cellular sites such as myoglobin, thus allowing continued ATP production via restoration of the mitochondrial membrane potential either directly by CCO activity or by activation of light or heat-gated ion channels. In addition to enhanced respiration via the ETC, relatively small concomitant increase in reactive oxygen spmayecies (ROS) production has been shown in vitro and vivo with light application.

ROS in healthy cells can provide enhanced cellular signaling and stimulation of nuclear transcription factors including nuclear transcription factor kappa beta, which can result in transcription of various proteins which can in turn provide cellular protection against oxidative stress caused by increased ROS. In exercising tissue, this upregulation of transcription factors may explain the chronic and long-term beneficial effects of PBM stemming from improved adaptation.

In various embodiments of the present inventive subject matter, light producing mechanisms are embedded in, or associated with, sportswear and clothing material and directed at specific muscle groups/skin sites to stimulate on a cellular, subcellular and systemic level the enhancement of muscular and exercise/activity performance. Activity oriented athletic wear directed to performance enhancement builds on solid scientific research both in vitro, on cells and isolated organelles, and in vivo in both animal and human trials. PBM has been shown to result in, but is not limited to, improvement in muscular performance, recovery, recovery markers on a biochemical level, and subjective user ratings. Though specific muscle groups are targeted and are benefited, there is research that supports improvement in systemic cardiovascular and pulmonary performance and biochemical measures of exercise and recovery.

In one embodiment, combinations or single LED's/LLL's are be embedded into sportswear and other wearable devices.

These light sources may have predetermined but adjustable parameters including but not limited to wavelength, pulse width, duration, dosage, and frequency. In at least some embodiments, each preselected wavelength of the light is selected to be at or near a transmission peak (or at or near an absorption minimum) for the intervening tissue. In at least some embodiments, one wavelength corresponds to a peak in the transmission spectrum of tissue at or near 850 nanometers (NIR). In at least some embodiments, one wavelength corresponds to a peak in the transmission spectrum of tissue at or near 650 nanometers (red visible).

Other specific embodiments use different means of light delivery, including OLEDs, microLEDs, or low-level lasers. Other specific embodiments also employ different means of embedding the light delivery mechanisms (e.g., LEDs) into the wearables, including glue, adhesive tape, snaps, stitching, heat activated seam tape, Velcro brand hook and loop fasteners, weaving the light delivery mechanisms into the fabric itself, or printing the light delivery mechanisms onto a flexible substrate embedded in or mounted on the fabric.

These parameters may be controlled via external and/or attached device by way of internal microprocessors and controllers. This external device could be but is not limited to smartphones, sports devices (e.g., sports watches or bike computers), or proprietary devices.

Parameters may be controlled manually or automatically based on the user's choice, but parameter values may be limited to be set within predefined safe ranges. In at least some embodiments, the temporal profile (e.g., peak irradiance, temporal pulse width, and duty cycle) is selected to utilize the kinetics of the biological processes while maintaining the irradiated portion of the tissue at or below a predetermined temperature.

In at least some embodiments, the pulse energy density, or energy density per pulse, can be calculated as the time-averaged power density divided by pulse repetition rate, or frequency. For example, the smallest pulse energy density will happen at the smallest average power density and fastest pulse repetition rate, where the pulse repetition rate is duty cycle divided by the temporal pulse width, and the largest pulse energy density will happen at the largest average power density and slowest pulse repetition rate. Conditional language, for example, among others, “can,” “could,” “might,” or “may,” unless specifically stated otherwise, or otherwise understood within the context as used, is generally intended to convey that certain embodiments include, while other embodiments do not include, certain features, elements or steps.

While the present disclosure has been discussed in the context of certain embodiments and examples, it should be appreciated that the present disclosure extends beyond the specifically disclosed embodiments to other alternative embodiments or uses of the present disclosures and obvious modifications and equivalents thereof.

Components can be added, removed, or rearranged. Additionally, the skilled artisan will recognize that any of the above-described methods can be carried out using any appropriate apparatus. Further, the disclosure herein of any particular feature, aspect, method, property, characteristic, quality, attribute, element, or the like in connection with various embodiments can be used in all other embodiments set forth herein. Additionally, processing steps may be added, removed, or reordered. A wide variety of designs and approaches are possible.

For purposes of this disclosure, certain aspects, advantages, and novel features of the present disclosure are described herein. It is to be understood that not necessarily all such advantages may be achieved in accordance with any particular embodiment of the present disclosure. Thus, for example, those skilled in the art will recognize that the present disclosure may be embodied or carried out in a manner that achieves one advantage or group of advantages as taught herein without necessarily achieving other advantages as may be taught or suggested herein.

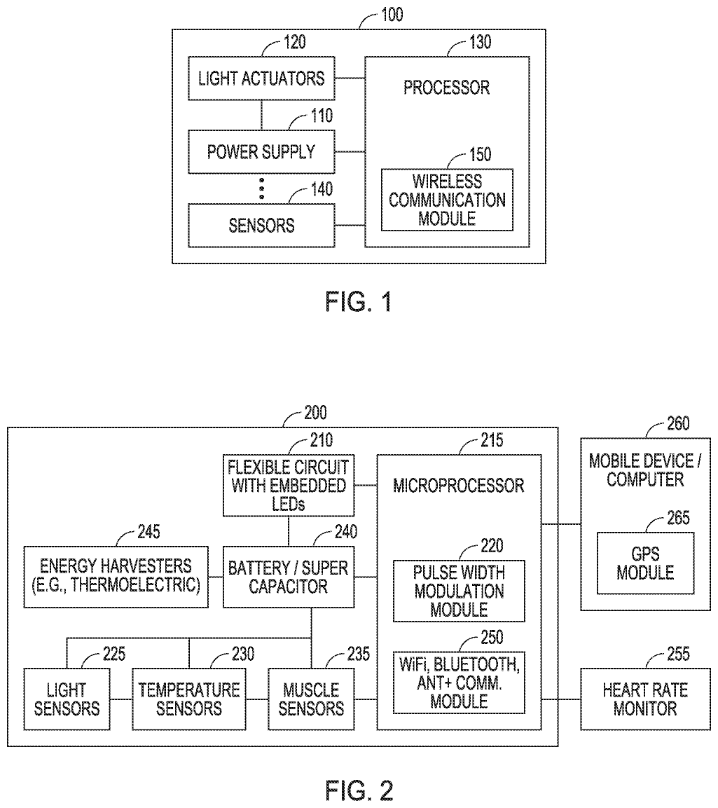

FIG. 1 is a block diagram of a system 100 using photobiomodulation for performance enhancement according to an example embodiment. In one embodiment, the system 100 is integrated or otherwise supported or held in place in a wearable. System 100 includes a power supply 110, one or more light sources, referred to as light actuators 120 and a controller 130 coupled to the power supply 110 and light actuators 120. The controller 130 controls the light actuators 120 to deliver light to targeted areas on the body as a function of the wearable position on the body.

The light actuators 120 can include an array of one or more LEDs, microLEDs, oLEDs, and LLL technology. The system 100 used an embedded controller 130, which may include a processor, such as a microprocessor that receives sensor readings via sensors 140, performs computations, controls actuators, and performs communication via wired or wireless communications module 150. Some functions of the controller 130 include control of pulse width modulation, dosimetry, spectral switching, communication with external devices such as phones, tablets, computers, smart watches, etc., data logging, interface with software.

Optional external or internal sensors 140 include, but are not limited to light sensor, EKG, power meter, lactate monitoring, muscle oxygen sensors, stretch sensor/strain gauge. A communication module is used to communicate with external devices or other wearable components. Communication protocols include but are not limited to Bluetooth, ant+, and/or Wi-Fi. Electrical components are ordered by the power supply 110, which may be a rechargeable battery, super-capacitor, and/or energy harvester. In some embodiments, the system 100 in the form of a wearable may have a rectangular shape that is large enough to provide light to selected parts of the body. The light actuators 120 may include an array of lights that may be controlled by the controller 130 to illuminate a selected body part by providing electricity to light actuators positioned about the selected part such that the light is directed to the selected body part. In further embodiments, arrays may be shaped to illuminate a selected body part with all light actuators providing illumination.

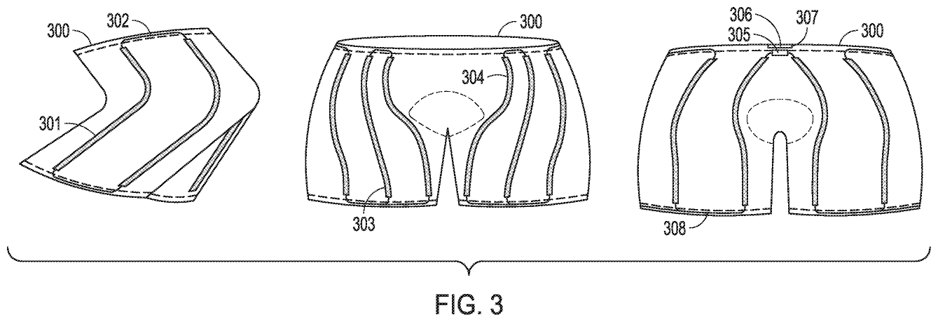

FIG. 2 is a block diagram illustrating a wearable item 200 having electronics and light actuators according to an example embodiment.

In one embodiment, wearable item 200 includes multiple components embedded into a wearable item such as a pair of shorts, a sleeve, etc. The embedded components may include LEDs of various wavelengths are mounted on a flexible circuit board or light delivery mechanism 210 that is attached to the wearable item 200 via stitching, snaps, glue, or Velcro brand hook and loop fasteners. The LEDs are controlled by an embedded microcontroller 215 that uses a pulse width modulation module 220 to control a power level and dose delivered by the LEDs. The microcontroller 215 also interfaces with various sensors 225, 230, 235 to determine appropriate dosing parameters—for example, muscle sensors 235 measure muscle contractions that help to determine optimal time between doses, temperature sensors 230 monitor temperature and prevent overheating, and light sensors 225 provide light measurement that may be used to adjust the applied dosage for various skin types and body compositions.

The electrical components embedded into the wearable are powered by an embedded battery or super-capacitor 240. In some embodiments, the battery/supercapacitor 240 is fed by energy harvesters 245 such as thermoelectric energy harvesters that harvest energy from surroundings and from the body itself to charge the battery/super-capacitor 240. The microcontroller 215 also contains a wireless communication module 250 that is capable of communication with external devices via Wi-Fi, Bluetooth, and ANT+ communication protocols. Examples of such compatible devices are heart rate monitors 255—used to monitor a user's heart rate to adjust applied dosage for the user's physical condition and exercise type—and mobile phones 260—used to communicate user inputs to the microcontroller 215 to control program parameters such as exercise type, exercise intensity, user characteristics, selection of manual or automatic dosing, desired dose levels and times, etc.

Communication with phone 260 that may have an embedded or external GPS modules 265 can also be used to gather information about user speed, distance traveled, and elevation traveled during exercise, which can be used to determine when and at what intensity to apply doses. Communication with mobile devices or computers can also be used to capture usage statistics, including time, intensity, and duration of applied doses. Other specific embodiments use different means of light delivery mechanism 210, including OLEDs, microLEDs, or low-level lasers. Other specific embodiments also employ different means of embedding the light delivery mechanisms 210 (e.g., LEDs) into the wearables, including glue, stitches, adhesive tape, snaps, hook and loop fasteners, weaving the light delivery mechanisms into the fabric itself, or printing the light delivery mechanisms 210 onto a flexible substrate embedded in or mounted on the fabric.

FIGS. 3A, 3B, and 3C are side, front, and back inside-out views of wearable shorts 300 incorporating a system for performance enhancement according to an example embodiment. The wearable shorts 300 may be compression/exercise shorts that deliver light to the lower body. Major components of the wearable include the light source 301 in the shape of LED strips or low-level lasers, conductors/wires 302 to connect electrical components, sensors 303 to modulate dosing, including but not limited to light sensor, lactate sensor, oxygen sensor, temperature sensor, or heart rate sensor, quick-connects 304 to allow easy removal of electronic components for garment laundering or component replacement, a microcontroller 305 to control sensing, actuation, and communication in the system, a battery 306 and/or supercapacitor for energy storage, energy harvester(s) 307 to increase battery lifetime, and a stretch sensor 308 to modulate dosing based on the surface area of the targeted region. In each of the wearables described herein, the light sources may be arranged to provide the ability to provide light to specific muscle groups that may be most involved in various selected physical activities. For runners, the light sources may include arrays of lights that are positioned along major muscle groups involved in running, or even smaller muscles that may contribute to high performance.

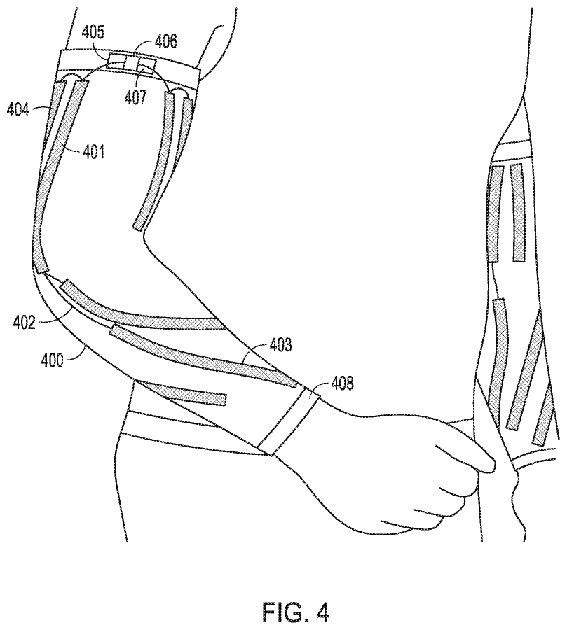

FIG. 4 illustrates a wearable sleeve 400 being worn and incorporating a system for performance enhancement according to an example embodiment. Sleeve 400 delivers light to an arm. Major components of the wearable include the light source 401 (e.g., LEDs or low-level lasers), conductors/wires 402 to connect electrical components, sensors 403, including light sensors to modulate dosing, quick-connects 404 to allow easy removal of electronic components for garment laundering or component replacement, a microcontroller 405 to control sensing, actuation, and communication in the system, a battery 406 and/or supercapacitor for energy storage, energy harvester(s) 407 to increase battery lifetime, and a stretch sensor 408 to modulate dosing based on the surface area of the targeted region. The light sources in sleeves may be arranged to provide light to muscles involved in racquet sports, with different arrangements for different types of racquet sports, such as racquet ball that uses the wrists more, while tennis may focus more on different parts of the arm, as snapping of the wrist is less prevalent. Golf may utilize still different muscles.

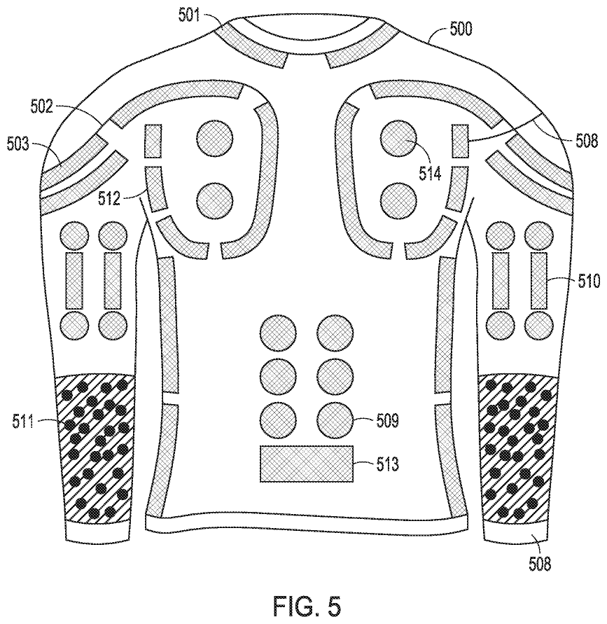

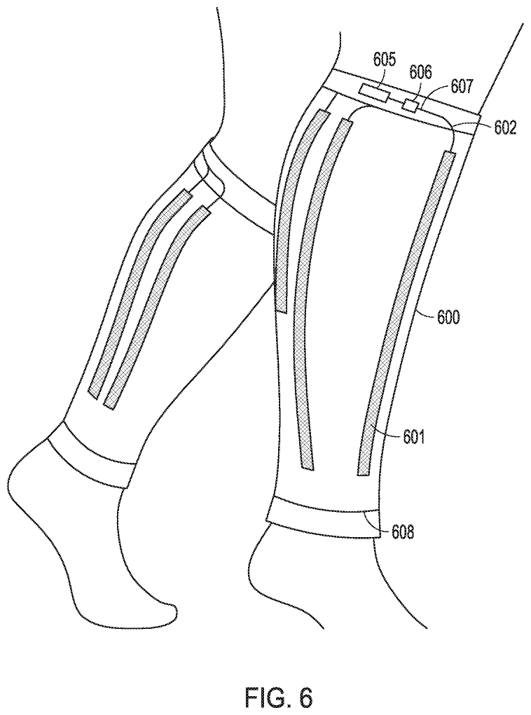

FIG. 5 illustrates an inside-out view of a wearable shirt 500 incorporating a system for performance enhancement according to an example embodiment. Shirt 500 is configured to deliver light to target areas throughout the upper body. Major components of the wearable include the light source (e.g., LEDs or low-level lasers) 501, conductors/wires 502 to connect electrical components, sensors 503, including light sensors to modulate dosing, stretch sensor(s) 508 to modulate dosing based on the surface area of the targeted region. A variety of light sources are available to fit different muscle groups/target areas, including multi-diode/multi-chipset pucks 509, multi-diode/multi-chipset strips 510 to provide concentrated light delivery to a target area such as biceps, printed micro LEDs on a form-fitting, flexible substrate 511, flexible oLED technology embedded in fabric 512, grid-embedded LED strips 513, and face generated low-level laser technology grids 514.

FIG. 6 illustrates a wearable sock 600 being worn and incorporating a system for performance enhancement according to an example embodiment. Sock 600 is configured to deliver light to the legs. Major components of the wearable sock 600 include the light source 601 (e.g., LEDs or low-level lasers), conductors/wires 602 to connect electrical components, sensors 604, including light sensors to modulate dosing, a battery 606 and/or supercapacitor for energy storage, energy harvester(s) 607 to increase battery lifetime, and a stretch sensor 608 to modulate dosing based on the surface area of the targeted region.

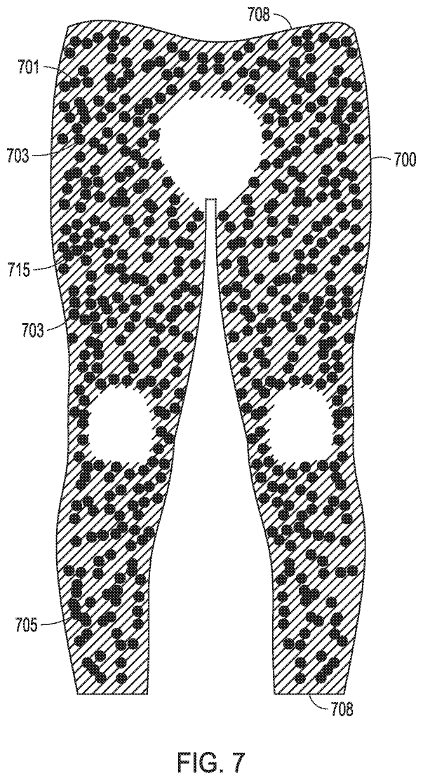

FIG. 7 illustrates a wearable pair of pants/tights 700 being worn and incorporating a system for performance enhancement according to an example embodiment. Pants/tights 700 may be configured to deliver light to target areas throughout the lower body. Major components of the wearable include the light source 701 (e.g., LEDs or low-level lasers), sensors 703, including light sensors to modulate dosing, stretch sensor(s) 708 to provide signals useful in modulating dosing based on the surface area of the targeted region. The light source incorporates a variety of technologies, including micro LEDs, oLEDs, grid-embedded LEDs, and face generated LLLT, to target a variety of bodily regions as indicated at 715. One embodiment may be produced by printing a network of conductive traces onto fabric and printing light sources and support circuitry onto the conductive network.

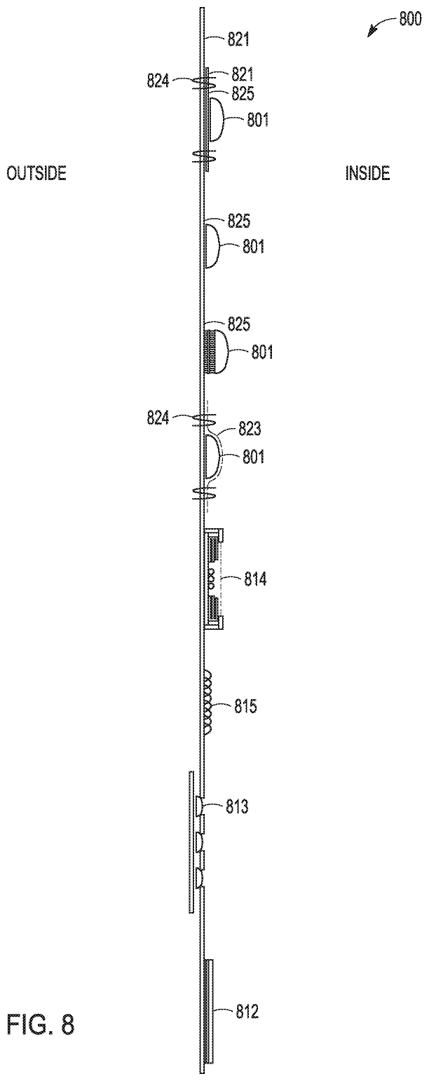

FIG. 8 is a block cross sectional representation 800 of multiple example wearable arrangements of light sources 801 supported by a substrate 821, such as a wearable material. Major components of the wearable layout are meant as a balance for cost, durability, performance, comfort, design, usability, waterproofing, washability, and appearance. These components include the light source 801 (e.g., LED strips or low level lasers), flexible oLED technology embedded in fabric 821, (13) grid embedded LED strips 813, and face-generated low-level laser technology grids 814. The light source 801 incorporates a variety of technologies, including micro LEDs, oLEDs, grid-embedded LEDs, and face-generated LLLT, to target a variety of bodily regions, substrate, eg. fabric 821, hook and loop connection 822, sheer fabric sleeve 823 for threading lights, stitching 824, e.g., thread, conductive thread, conductive ink, conductive epoxy, conductive solder, flexible thread, adhesive 825, e.g., tape, glue, heat, fusion, deposition, or other means for conducting electricity to the light sources.

The following list of components may be incorporated into one or more wearables. Some of the components are shown in FIG. 8 with reference numbers.
801—light source in the red/near ir wavelength
conductor/wiring/thread/elastic, conductive ink, conductive epoxy, conductive solder.
light sensor/other sensors
quick connect
microcontroller
battery or super capacitor
energy harvester
stretch sensor
multidiode/multichipset puck
multidiode/multichipset strip
printed flexible microLED—red/near ir wavelength
812—oLED fabric —red/near ir wavelength—same as claim one
813—grid embedded LED—red/near ir wavelength—same as claim one
814—Face generated LLLT—red/near ir wavelength—same as claim one
815—microLED, OLED, grid embedded, Face gen LLLT red/near ir wavelength—same as claim one [0063]
wearable device shown ‘inside out’ (faces reversed)
821—substrate
hook and loop connection
sheer fabric sleeve for threading lights
824—stitching
825—adhesive

Representation 800 illustrates various groupings from top to bottom of components on substrate 821. A first grouping includes stitching 824, adhesive 825, and light source 801. A next grouping just includes light source 801 supported on substrate 821 by adhesive 825. Note that each wearable item may have multiple of one type of grouping in an array, or a mixture of groupings.

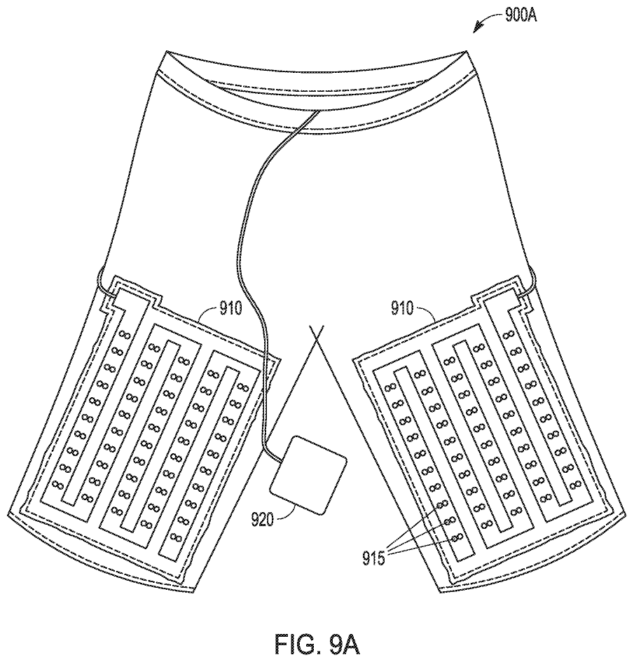

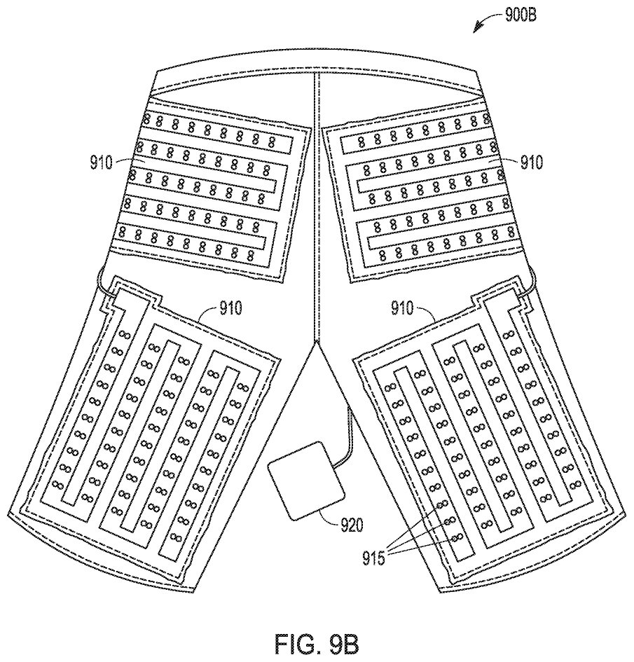

FIG. 9 is a block schematic diagram illustrating an inside-out front view of a prototype pair of shorts 900 having a substrate 910 supporting an array of light sources 915 on each leg. The substrate 910 may be referred to as panels. The light sources 915 are arranged in strips to illuminate the major muscle groups on the front of the leg of a wearer.

In various embodiments, the panels are made up from a sandwich of materials that are heat pressed together and sewn into the shorts 900. The material that is pressed onto the LED light sources 915 is first laser cut to the approximate to exact opening of the diodes and provides a layer of waterproofing and utilizes a glue that is activated by heat and pressure or ultrasonic waves. In further embodiments a UV cured adhesive may be used.

Wiring options may include a flexible wiring solution that sews/presses the wiring into a tunnel system in one iteration. In a further embodiment, a sinusoidal wire layout is created and pressed together that allows for stretching with the movement of the body.

Wiring connections may be established with quick connects, solder joints, and a mechanically strain relieved with both pressing and custom 3D printed parts. A micro controller and battery box 920 in this version is 3D printed with a flexible material and snap enclosure. A pocket on a back side of the shorts 900 may be used to hold the box 920. A further option for box 920 includes the use of metal snaps to provide power and communicate signals to the box.

FIG. 9B is an inside-out back view of the shorts 900. Four substrates 910 supporting light source arrays 915 are illustrated. Two substrates 901 are positioned to illuminate the buttocks and two to illuminate the back of the legs of the wearer are shown.

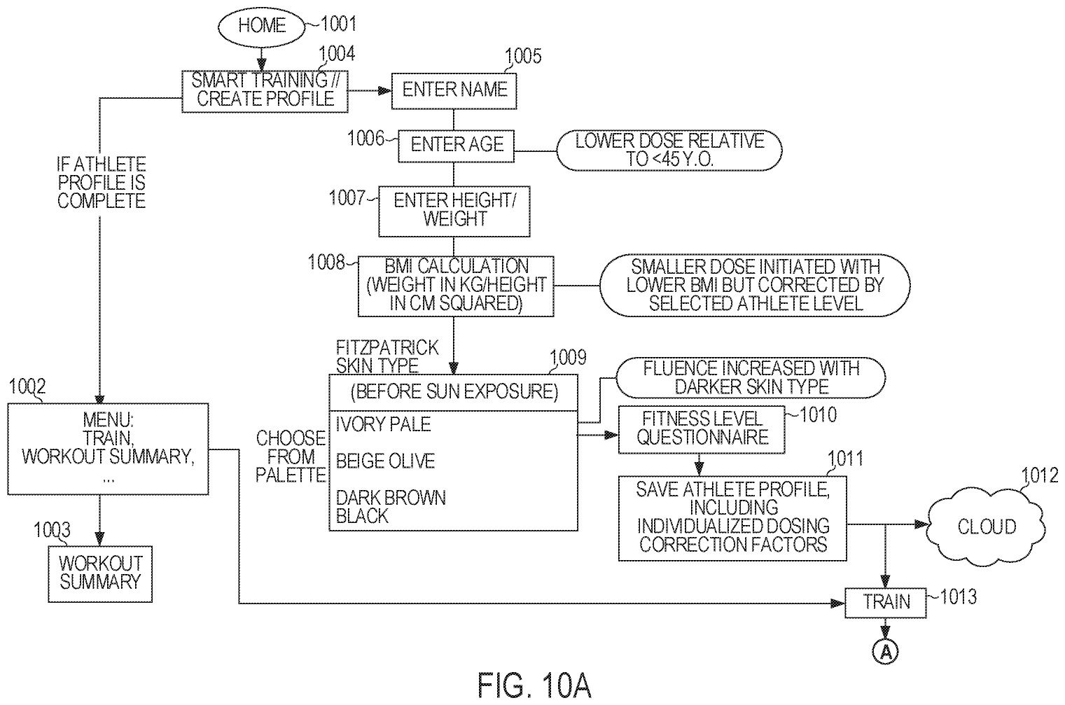

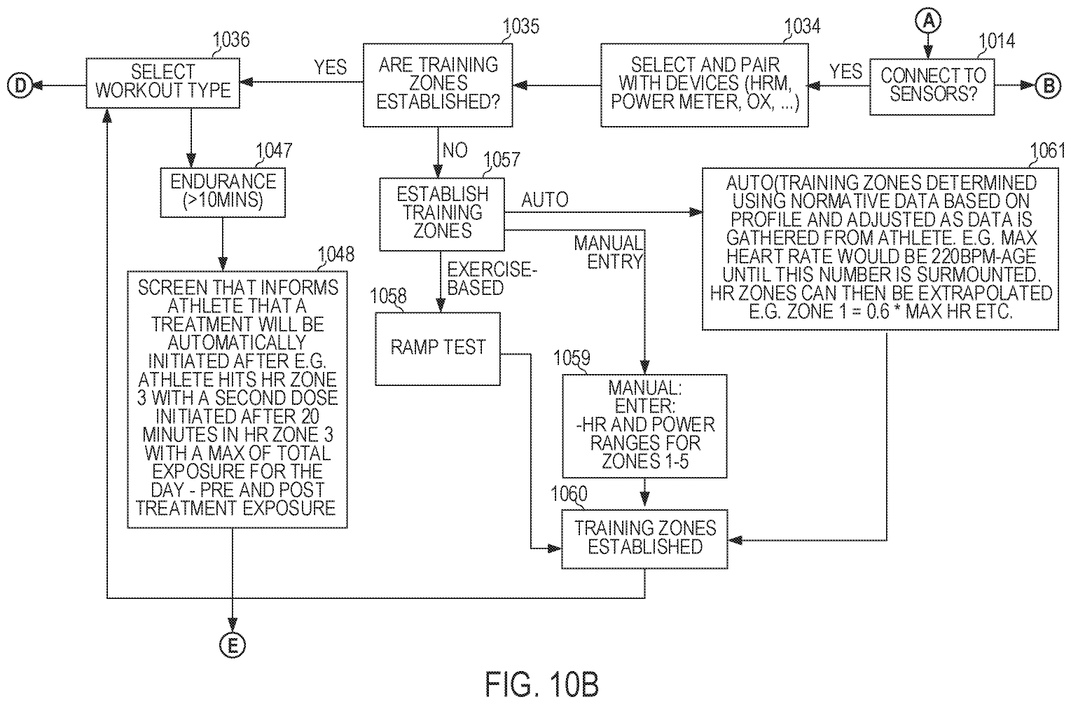

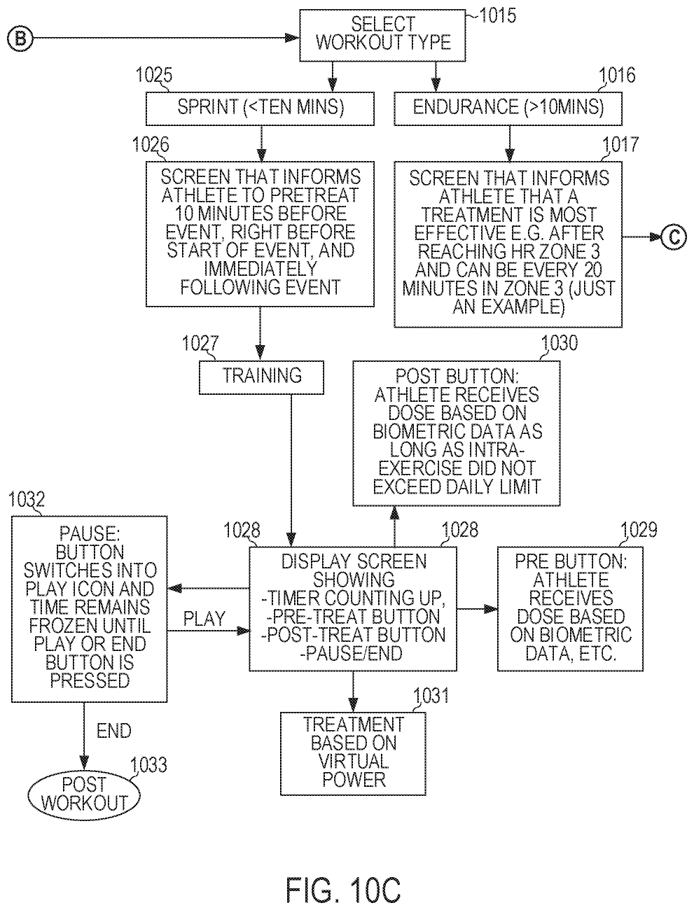

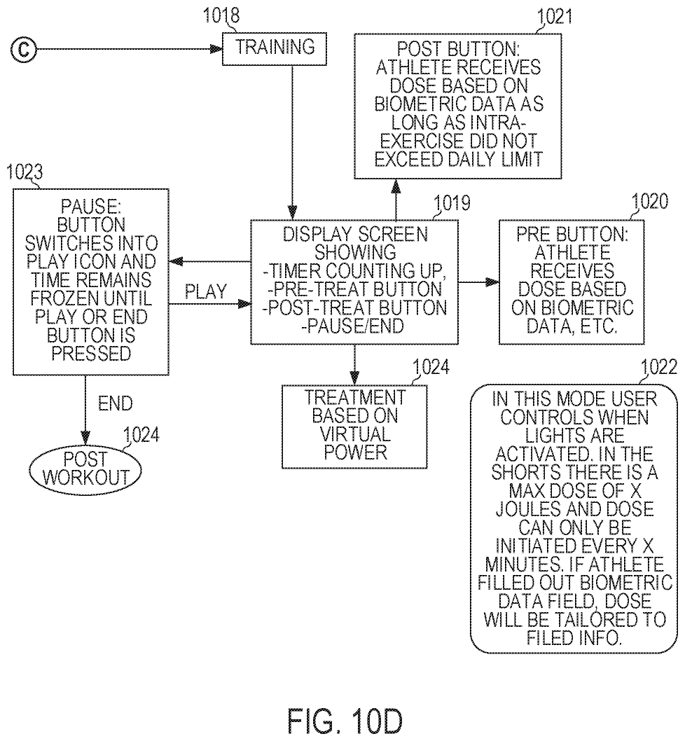

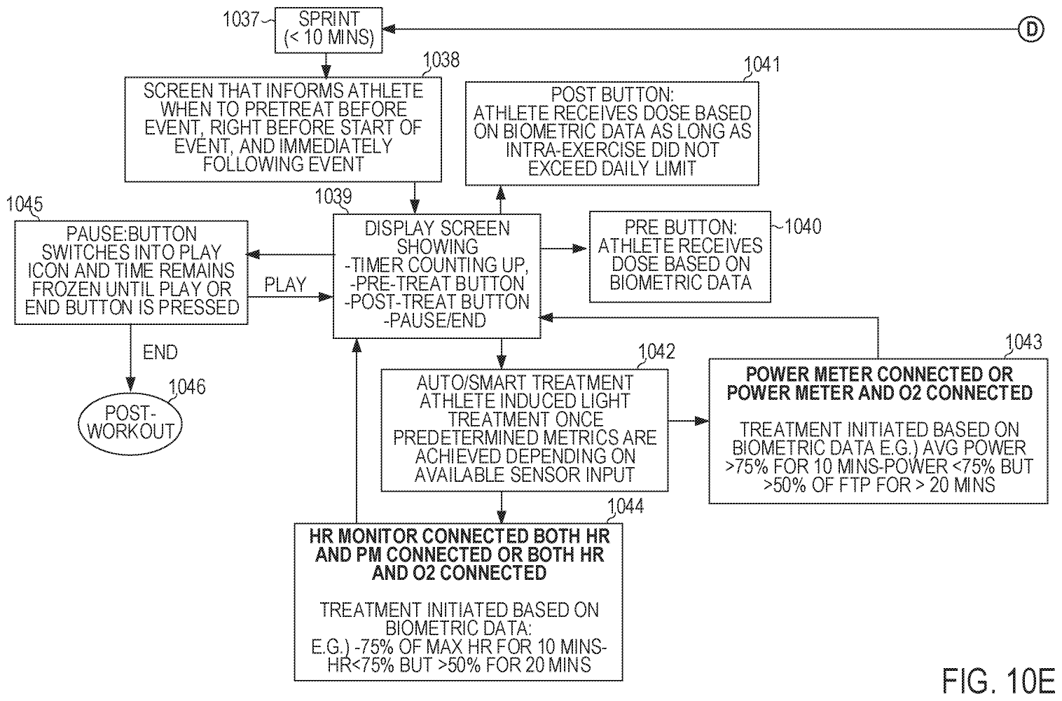

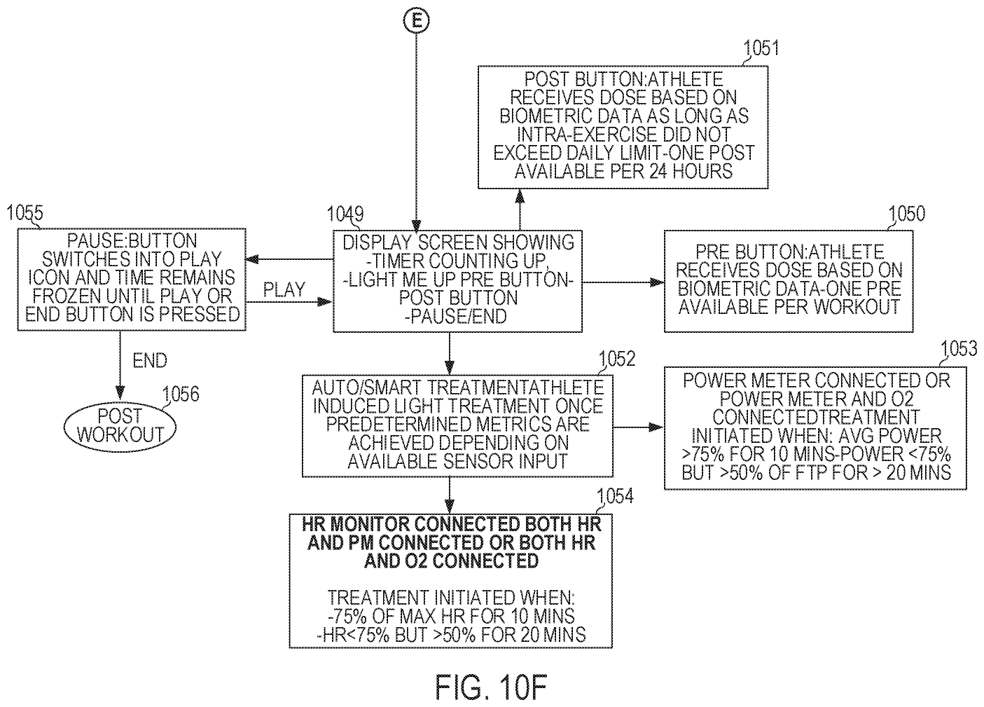

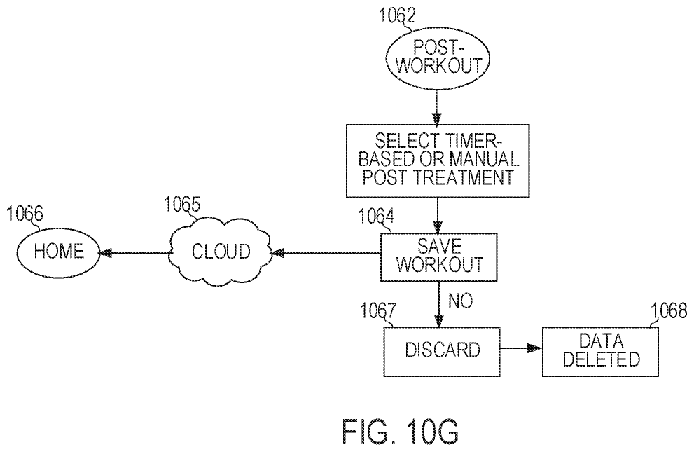

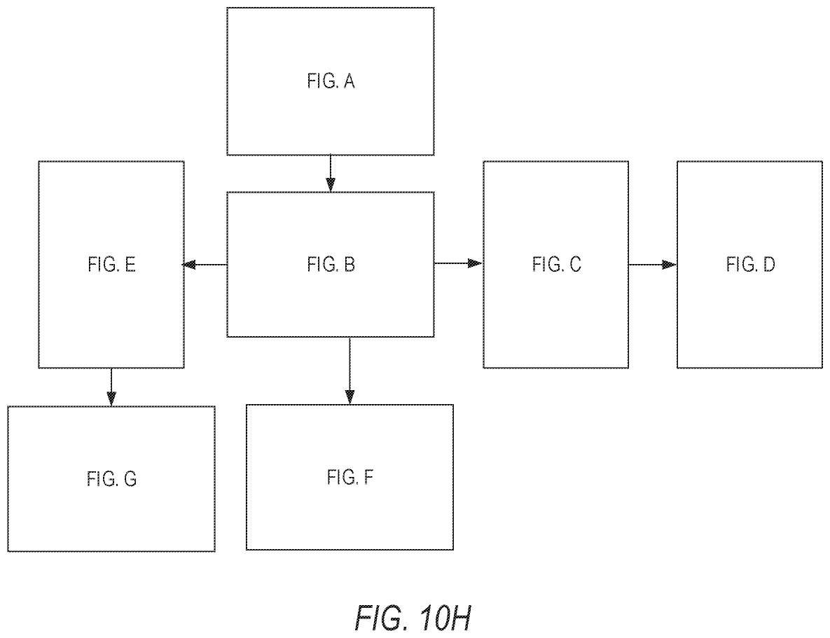

FIGS. 10A, 10B, 10C, 10D, 10E, 10F, and 10G are a flowchart illustrating a computer implemented method 1000 use and operation of a wearable, such as a garment for enhancing performance according to an example embodiment. Starting at home 1001, if a profile of the user, such as an athlete or wearer of the garment is complete, the user is provided a menu for selecting from options, such as training or working out. A workout summary 1003 is also available to show data from a workout, such as sensor data, light dose information, duration, and other information.

If there is no profile, a create profile operation 1004 is executed to prompt a user to enter a name at 1005, enter an age at 1006, enter a height and weight at 1007, BMI 1008, and a skin type at 1009. The skin type may be a Fitzpatrick skin type and a palate of ivory pale, beige olive, and dark Brown/Black may be selected, and based on type before sun exposure. Lower doses of light may be provided for users less than a certain age, such as 45 years. Smaller doses may be used with lower body mass index (BMI), but may be modified based on fitness level. Doses may be increased with darker skin types. A fitness level questionnaire may be provided at 1010 to determine the athletic level. The profile is saved at 1011 and may be stored in cloud based resources 1012.

The user is then ready to train at operation 1013. A first check at operation 1014 is made to determine whether any sensors are associated with the wearable. If not, the user is prompted to select a workout type at operation 1015. In one example, if the user selects an endurance type of workout, say greater than 10 minutes in length of example as indicated at 1016, a screen is provided at 1017 that informs the user that a treatment is most effective after reaching a target heart rate (HR) zone 3 and can be every 20 minutes in zone 3, just as an example. Training begins at 1018. A display screen may be provided at 1019 to show a timer counting up, a pre treat button, a post treat button and a pause/end button. Selection of pre treat is illustrated at 1020 where the user receives a dose based on biometric data obtained from the user profile. Selection of post treat is illustrated at 1021 where the user receives a dose of light based on biometric data as long as intra-exercise and not having exceeded a daily limit. At operation 1022 the light dose may be based on a perception of virtual power. The virtual power can be estimated based on the information in the user profile, including parameters such as weight, height, fitness level, and individualized correction factors. The pause/end button leads to operation 1023 where the user may select play to resume training or end to end the workout and lead to post workout operations as indicated at 1024. In the endurance training mode, the user controls when light sources are activated. If the garment is a pair of shorts, there may be a maximum does of X joules and activation may occur every X minutes. If the user filled out the biometric data, the does may be tailored to the stored user/athlete profile.

If at 1015 the user selected “sprint” for the workout type, defined in one example as a less than 10 minute workout at 1025, the user is provided options similar to those provided for the endurance workout type. A screen 1026 may be provided to informs the user to pretreat 10 minutes before training, right before starting, and immediately following training, referred to as “the event.”

Training may begin as indicated at operation 1027, resulting in the similar display screen 1028 showing timer and pre and post buttons as well as a pause/end button. Selection of pre treat is illustrated at 1029 where the user receives a dose based on biometric data obtained from the user profile. Selection of post treat is illustrated at 1030 where the user receives a dose of light based on biometric data as long as intra-exercise and not having exceeded a daily limit. At operation 1031 the light dose may be based on a perception of virtual power. The virtual power can be estimated based on the information in the user profile, including parameters such as weight, height, fitness level, and individualized correction factors. The pause/end button leads to operation 1032 where the user may select play to resume training or end to end the workout and lead to post workout operations as indicated at 1033.

At operation 1014, if sensors are associated with the wearable, pairing of the sensors with the controller may be performed at operation 1034. Typical sensors include HR monitor, power meter, oxygen sensor, and others. In one example, operation 1035 determines if training zones have been established. If yes, a workout type may be selected at operation 1036. If a sprint workout is selected as indicated at 1037, a screen 1038 may be provided to informs the user to pretreat 10 minutes before training, right before starting, and immediately following training, referred to as “the event.” Training may begin, resulting in the similar display screen 1039 showing timer and pre and post buttons as well as a pause/end button. At this point, the operations are modified from the modes without the use of sensors. At operation 1040 the user receives a dose based on biometric data upon selection of the pre button. Selection of the post button results in post training treatment operation 1041 where the user receives a dose based on biometric data as long as intra-exercise did not exceed a daily limit. At operation 1042 an automatic dosing occurs during training once predetermined metrics (sensed parameter levels) are achieved depending on available sensor input. In one example, the sensor input may include data from a power meter or oxygen sensor as indicated at operation 1043, resulting in treatment being initiated based on biometric data. For example, light doses may be provided once the average power has exceeded 75 percent of the maximum power for greater than 10 minutes for a user as derived from the user profile. In one embodiment, doses are provided in response to avg power>75% for 10 mins-Power<75% but >50% of FTP for >20 mins. Similarly, data from an HR monitor and or PM and O2 sensor may be used as indicated at operation 1044. Treatment initiated based on biometric data: e.g.) −75% of max HR for 10 mins-HR<75% but >50% for 20 mins. Selection of pause leads to operation 1023 where the user may select play to resume training or end to end the workout and lead to post workout operations as indicated at 1024.

If at operation 1036, the workout type “endurance” was selected as indicated at 1047 leads to operation 1048 and a Screen that informs athlete that a treatment will be automatically initiated after e.g. athlete hits HR zone 3 with a second dose initiated after 20 minutes in HR zone 3 with a max of Total exposure for the day—Pre and post treatment exposure. This is followed by a screen at operation 1049 showing the timer and pre, post, and pause/end buttons. In response to the pre button, the user receives a dose based on biometric data at operation 1050. One pre dose may be provided per workout/training session. Selection of the post button results in operation 1051 where the user receives a dose based on biometric data as long as intr-exercise did not exceed a daily limit. One post dose may be available per 24 hours in one embodiment. During the workout, an auto/smart dosing may be provided via operation 1052 once predetermined metrics are achieved depending on available sensor input. For example, operations 1053 and 1054 may function in a manner similar to operations 1043 and 1044 based on power and HR monitors/sensors respectively. Selection of pause leads to operation 1055 where the user may select play to resume training or end to end the workout and lead to post workout operations as indicated at 1056.

If at operation 1035, training zones have not been established, operation 1057 is used to establish training zones in one of several different ways. One way includes a ramp test at operation 1058 where the user basically begins a workout and ramps up effort. Parameters may be measured during the ramp up and used to establish the training zones by comparing the measured parameters to parameters associated with known zones to select a corresponding zone. At operation 1059, a user may manually enter HR and power ranges for zones such as zones 1-5. At operation 1061, training zones may be automatically determined using normative data based on user profile and adjusted as data is gathered from athlete. e.g. max heart rate would be 220 bpm-age until this number is surmounted. HR zones can then be extrapolated e.g. Zone 1=0.6*Max HR etc. Once one of these methods are completed, the training zones are established as indicated at operation 1060.

The post workout module begins at operation 1062. The module is entered each time one of the preceding workout/training modes is ended by the user. Post treatment may be performed at operation 1063 based on a timer or a manual post treatment selection by the user. Operation 1064 may be used to save the workout to cloud based resources at 1065 followed by a return to home 1067. The user may also select to discard the workout at 1067, resulting in the data associated with the workout being deleted at operation 1068.

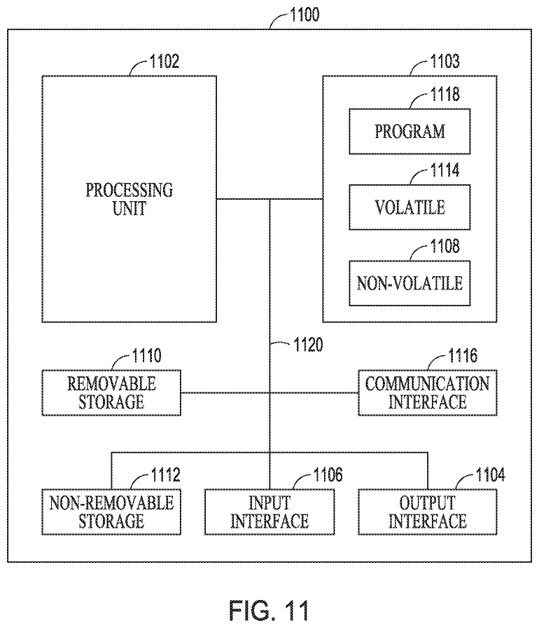

FIG. 11 is a block diagram illustrating circuitry for controlling lights to enhance performance of selected muscle groups and performing methods according to example embodiments. All components need not be used in various embodiments.

One example computing device in the form of a computer 1100 may include a processing unit 1102, memory 1103, removable storage 1110, and non-removable storage 1112. Although the example computing device is illustrated and described as computer 1100, the computing device may be in different forms in different embodiments. For example, the computing device may instead be a smartphone, a tablet, smartwatch, or other computing device including the same or similar elements as illustrated and described with regard to FIG. 11. Devices, such as smartphones, tablets, and smartwatches, are generally collectively referred to as mobile devices or user equipment. Such devices may be worn separately from, or integrated into the wearable device incorporating light delivering devices. Further, although the various data storage elements are illustrated as part of the computer 1100, the storage may also or alternatively include cloud-based storage accessible via a network, such as the Internet or server based storage.

Memory 1103 may include volatile memory 1114 and non-volatile memory 1109. Computer 1100 may include—or have access to a computing environment that includes—a variety of computer-readable media, such as volatile memory 1114 and non-volatile memory 1108, removable storage 1110 and non-removable storage 1112. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 1100 may include or have access to a computing environment that includes input interface 1106, output interface 1104, and a communication interface 1116. Output interface 1104 may include a display device, such as a touchscreen, that also may serve as an input device. The input interface 1106 may include one or more of a touchscreen, touchpad, mouse, keyboard, camera, one or more device-specific buttons, one or more sensors integrated within or coupled via wired or wireless data connections to the computer 1100, and other input devices. The computer may operate in a networked environment using a communication connection to connect to one or more remote computers, such as database servers. The remote computer may include a personal computer (PC), server, router, network PC, a peer device or other common DFD network switch, or the like. The communication connection may include a Local Area Network (LAN), a Wide Area Network (WAN), cellular, WiFi, Bluetooth, or other networks. According to one embodiment, the various components of computer 1100 are connected with a system bus 1120.

Computer-readable instructions stored on a computer-readable medium are executable by the processing unit 1102 of the computer 1100, such as a program 1118. The program 1118 in some embodiments comprises software that, when executed by the processing unit 1102, performs operations according to any of the embodiments included herein. A hard drive, CD-ROM, and RAM are some examples of articles including a non-transitory computer-readable medium such as a storage device. The terms computer-readable medium and storage device do not include carrier waves to the extent carrier waves are deemed too transitory. Storage can also include networked storage, such as a storage area network (SAN). Computer program 1118 may be used to cause processing unit 1102 to perform one or more methods or algorithms described herein.

### EXAMPLES

1. A method comprising: applying a substrate containing light emitting devices to a body part; and controlling the light emitting devices to deliver athletic performance enhancing light to the body part, wherein the light has a wavelength in the range of 600 nm to 1300 nm.

2. A method comprising: applying a substrate containing light emitting devices to a body part; and controlling the light emitting devices to deliver athletic performance enhancing light to the body part, wherein the light has a wavelength in the range of 400 nm to 1300 nm.

3. A performance-enhancing wearable device and method that delivers light in the red and/or near-infrared spectrum in the range of 600 nm-1300 nm, which may be used concurrently or at different time periods and the Red/IR light may be controlled independently.

4. The method of any of the previous examples wherein the red light is delivered at an irradiance from 2 mW-600 mW/cm2

5. The method of any of the previous examples wherein the red light is delivered at an irradiance of at least 5 mW/cm2

6. The method of any of the previous examples wherein the red light is delivered at an irradiance of at least 10 mW/cm2

7. The method of any of the previous examples wherein the near infrared light is delivered at an irradiance from 2 mW-600 mW/cm2.

8. The method of any of the previous examples wherein the near infrared light is delivered at an irradiance of at least 5 mW/cm2.

9. The method of any of the previous examples wherein the near infrared light is delivered at an irradiance of at least 10 mW/cm2.

10. The method of any of the previous examples wherein the total light dose is limited to only that which continues to provide improved performance enhancement and/or recovery as determined by the biphasic dose response curve and thermal relaxation time. One example of a biphasic dose response includes: A light dose of less than X joules does not trigger performance enhancement; light dose between X and Y joules triggers performance enhancement; light dose above Y joules is detrimental to performance enhancement. X and Y may be determined empirically.

11. The method of any of the previous examples wherein the light dose is controlled in accordance with a protocol that is used to capture an individual's characteristics such as skin color, body type, body mass index, reflectance, and current fitness level. Skin color, skin thickness, subcutaneous fat affect absorption and reflection of light. Therefore, the total dose delivered is adjusted to account for the effect of these parameters on light absorption and reflection. As fitness level increases, mitochondrial density increases. Therefore, more light energy can be delivered without exceeding the upper limit on the biphasic dose curve.

12. The method in example 11, wherein an individual's dose is higher if the individual's skin color is darker, e.g. darker skin may require higher irradiance than a lighter skin individual depending on the given light wavelength. A certain amount of energy is referred to as a dose. The length of time required to deliver a certain amount of energy depends on the power output of the lights (irradiance).

13. The method of any of the previous examples wherein the device is attached to or embedded into sportswear or clothing in close proximity to an individual's target tissue or body area.

14. The method of any of the previous examples wherein the light source is held in close proximity to an individual's target tissue with one or more of stitching, hook and loop fasteners, magnetic strips, adhesive, heat activated seam tape, compression, and embedded within the textile itself

15. The method of any of the previous examples wherein the light source is held at a near constant distance between the light source and the skin and aided by a thin fabric or film between the users skin and light source

16. The method of any of the previous examples wherein a well-trained elite athlete may be provided higher dose per area than the dose given to a less well-trained athlete (given the higher overall muscle/vasculature density)

17. The method of any of the previous examples, wherein the controller is be set to treat at pre-specified parameters for prolonged use.

18. The method of any of the previous examples, where an onboard microcontroller is in sync with an external device such as power meter, muscle oxygen sensor, lactate monitor, stretch or strain gauge sensor, or heart rate monitor, light therapy would be delivered when a certain physiologic threshold is reached, for example, heart rate greater than 150 beats per minute activates the lights automatically.

19. The method of any of the previous examples, wherein a power supply, such as a battery, super-capacitor, or energy harvester is integrated into or affixed to the clothing, be chargeable, and also removable to allow cleansing of garment

20. The method of any of the previous examples wherein the light source is low level light therapy, which is selectively pulsed, super-pulsed, continuous, or applied sequentially to target tissue as determined by activity goals (recovery vs increased performance), activity, and physical state of the participant

21. The method of any of the previous examples, wherein the target tissue is otherwise healthy and is not in a diseased state other than currently active and in an exercised state.

22. The method of any of the previous examples, wherein the light source is in close proximity and targets related chromophores in but not limited to the epidermis and associated appendages, reticular and papillary dermis, blood vessels in the dermis and other subcutaneous tissues, fascial plane and associated tissue, hematologic cells, as well as skeletal muscle and associated cells and blood vessels.

23. The method of any of the previous examples, wherein the goal of target tissue being treated with various wavelengths potentially induces a measurable performance enhancement including but not limited to biochemical markers such as creatinine kinase, lactate and lactic acid, and other breakdown byproducts, nitrite-nitric oxide modulation, cytochrome C oxidase and other mitochondrial proteins, delayed gene transcription by way of oxidative stress, as well as other targets in the tissue.

24. The method of any of the previous examples, wherein the goal of target tissue response is:

Increased muscular performance and/or

Decreased recovery time and/or

Decreased perceived post or intra-exercise pain or fatigue and/or

Decreased delayed onset muscle soreness and/or

Enhanced psychological perception of exercise.

25. The method of any of the previous examples, wherein the light therapy is applied shortly before, and/or during and/or after exercise at the pre-specified dosage parameters.

26. The method of any of the previous examples, wherein the wearable is activity clothing including but not limited to: compression shorts, biking shorts, socks, leggings, underwear, hat, sports tape, wristband, sleeve, jersey, full body suit, shirt, any other iteration with wearable light source for performance/activity enhancement.

27. The method of any of the previous examples, wherein the therapy parameters are adjusted or preset prior to an activity, or adjustable via a separate microprocessor or controller and limited/controlled by a maximum amount of energy delivered in a given time period and time interval between light applications.

28. The method in example 27, wherein the light parameters are adjusted and monitored by devices such as external application on a smart device, work out computer (e.g, activity watch or bike computer), and/or interface with heart rate monitor, power meter or a proprietary device.

29. The method of any of the previous examples, wherein the lights are encased into a reflective shield to allow unidirectional light application to the skin and prevent unnecessary treatment to other body parts including the eye.

30. The method of any of the previous examples, wherein the performance enhancing device may be modified by application of topical lotion, cream, gel, ointment, or fluid that could include topical Nitric oxide stimulators, reactive oxygen scavengers, creatine, or electron transport chain enhancers.

Nitric oxide stimulators could include nitric oxide precursors in the nitric oxide stimulator pathway.

Reactive oxygen scavengers could include vitamins with antioxidant activity, tetrahydrocurcumin, curcumin.

Creatine to allow adequate storage and precursors for the ATP system.

Niacinamide for adequate storage/modulation of the precursor molecule NADH.

31. The method of any of the previous examples, wherein sensors are utilized to monitor cutaneous temperature to provide automated discontinuation if pre-specified temperatures are reached.

32. The method of any of the previous examples, wherein the calculated safe and optimal dose is measured by integrated stretch sensors, which may be used to calculate the surface area of the treated area is determined.

33. The method of any of the previous examples wherein the parameters of light delivery can be adjusted by a processing unit

34. The method of any of the previous examples wherein the amount of energy delivered is determined by a processing unit to account for number of light sources, surface area, battery level, and/or user variability

35. The method of any of the previous examples, wherein a local network is created between multiple garments to coordinate light application.

35A. The method of example 35 wherein the local network comprises a mesh network.

36. The method of any of the previous examples, wherein garments containing light emitting devices are enhanced for durability, including but not limited to “ease” in the attachment between light emitting devices and garment to prevent stress on circuitry.

37. The method of any of the previous examples, wherein the substrate is a flexible, compressible, stretchable substrate adjoined by stretchable wiring to prevent stress on wires and connections, which may include stretchable stitches and/or stress relief features between circuitry and wiring and/or seam tape for water resistance and protection of circuitry and injury protection and/or optimize goniometry to personalize garment fit for individual users.

38. The method of any of the previous examples, wherein a panel design of lights is affixed to the garment separately. The panel design includes heat activated seam tape to compression flexible fabric that is stitched to the garment separately to allow flexibility of the panel and relieve mechanical stress on the light device.

39. The individual dosing parameters of the collective data from activity and performance can be logged and uploaded to a database for future use.

40. A performance-enhancing wearable device comprising: a substrate adapted to be worn proximate a body part whose athletic performance is to be enhanced; an array of light producing elements positioned to direct light to the body part, and held in place by the substrate; and a controller to control the array of light producing elements to provide performance enhancing light to the body part, wherein the light has a wavelength in the range of 400 nm to 1300 nm.

41. The device of example 36 wherein the substrate comprises clothing.

46. The device of example 36 and further comprising at least one of an external or internal sensor, including power meter, lactate, oxygen sensor, strain gauge, heart rate monitor coupled to the controller.

47. The device of example 36 wherein the controller controls less than all of the light producing element to provide light corresponding to a shape of the body part.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. A method comprising:
applying a substrate containing light emitting devices to an individual's body part; and
controlling the light emitting devices to deliver athletic performance enhancing light to the body part, wherein the light has a wavelength in the range of 400 nm to 1300 nm.

2. The method of claim 1 wherein the light has a wavelength in the range of 600 nm to 1300 nm.

3. The method of claim 1 wherein the red light s delivered at an irradiance from 2 mW-600 mW/cm2

4. The method of claim 1 wherein the red light is delivered at an irradiance of at least 5 mW/cm2.

5. The method of claim 1 wherein the total light dose is limited to that which continues to provide improved performance enhancement and/or recovery as determined by a biphasic dose response curve and thermal relaxation time.

6. The method of claim 1 wherein the light dose is controlled based on the individual's characteristics comprising one or more of skin color, body type, body mass index, reflectance, and current fitness level.

7. The method of claim 6, wherein the individual's dose is higher if the individual's skin color is darker, wherein the dose is determined as a function of irradiance power, wavelength, and length of time.

8. The method of claim 1 wherein the light emitting devices are attached to or embedded into sportswear or clothing in close proximity to the individual's body part comprising target tissue or body area.

9. The method of claim 8 wherein the light emitting devices are held in close proximity to the individual's target tissue with one or more of stitching, hook and loop fasteners, magnetic strips, adhesive, heat activated seam tape, compression, and embedding within the garment itself.

10. A performance-enhancing wearable device comprising:
a garment adapted to be worn by a user;
one or more substrates supported by the garment;
one or more light sources supported by the one or more substrates and positioned on the garment to provide light in the red and/or near-infrared spectrum in the range of 600 nm-1300 nm to selected portions of the user wearing the garment;
a power source to provide power; and
a controller coupled to the one or more light sources and power source to controllably cause the one or more light sources to provide the light.

11. The device of claim 10 wherein the one or more light sources are independently controlled by the controller to provide light at different time periods.

12. The device of claim 10 wherein the one or more light sources are attached to, or embedded into a garment comprising sportswear or clothing in close proximity to the individual's body part comprising a target tissue or target body area.

13. The device of claim 12 wherein the one or more light sources are held in close proximity to the individual's target tissue with one or more of stitching, hook and loop fasteners, magnetic strips, adhesive, heat activated seam tape, compression, and embedding within the garment itself.

14. A machine-readable storage device having instructions for execution by a processor of a machine to cause the processor to perform operations to perform a method of applying performance enhancing light via a garment supporting light emitting devices positioned to direct light to a selected body part of a wearer of the garment, the operations comprising:
controlling the light emitting devices to deliver athletic performance enhancing light to the body part; and
wherein the light is controlled to have a wavelength in the range of 400 nm to 1300 nm and an irradiance of from 2 mW-600 mW/cm2.

15. The machine-readable storage device of claim 14 wherein the total light dose is limited to that which continues to provide improved performance enhancement and/or recovery as determined by a biphasic dose response curve and thermal relaxation time.
